(19) United States
(12) Plant Patent Application Publication
(10) Pub. No.: US 2019/0394917 P1
(43) Pub. Date: Dec. 26, 2019

(54) Rhododenron plant named kurachouchou

(50) ;

(71) Applicant: Kabushiki Gaisha Akatsuka Garden Co., Ltd., Tsu-shi (JP)

(72) Inventor: Yukio Kurabayashi, Tsu-shi (JP)

(21) Appl. No.: 16/501,341

(22) Filed: Mar. 28, 2019

(30) Foreign Application Priority Data
Jun. 20, 2018 (JP) 33199

(51) Int. Cl. A01H 6/36

(52) U.S. Cl. PLT/238

(57) ABSTRACT

The present invention relates to a new and distinct variety of plant named ‘Kurachouchou.’ The variety is botanically identified as Rhododendron L. The new variety is distinguished from other varieties by a number of properties, including but not limited to, corolla lobe color.

Latin name of the plant claimed: The variety is botanically identified as Rhododendron L.

### CROSS-REFERENCE TO RELATED APPLICATION

This application claims benefit of Japanese Plant Variety Protection Application No. 33199, filed on Jun. 20, 2018, which is incorporated herein in its entirety.

### BACKGROUND OF THE INVENTION

The new variety ‘Kurachouchou’ originated from the controlled cross of the plants ‘Stardust’ and ‘Pink Ice’ performed in April 2012. ‘Kurachouchou’ were planted in the fall of 2012 in Anoucho, Tsu-shi, Mie-ken, Japan. From among the flowers that bloomed in 2015, those that were late blooming with attractive pink rims were selected. As a result of tissue cultures in 2015 and trial production in the field, ‘Kurachouchou’ was determined as being a practical variety in May 2018, and breeding was completed.

### SUMMARY

The present invention relates to a new and distinct variety of plant named ‘Kurachouchou.’ The variety is botanically identified as Rhododendron L. The new variety is distinguished from other varieties by a number of characteristics as set forth below.

When compared to the ‘Mrs. Fujii’ variety having blossoms with a primary color that is pale pink, almost white (RHS color chart N74D) and a secondary color that is a very pale purple (RHS color chart 76D), the new variety of the present invention has been found to exhibit attractive blossoms with a principle strong purplish red color (RHS color chart 71C) and a secondary color that is very light purple (RHS color chart 76B). The annual blooming time for ‘Kurachouchou’ is one month later than ‘Mrs. Fujii.’ ‘Kurachouchou’ has larger leaves (about 11 centimeters (cm) in length and about 4.0 cm in width) compared to ‘Mrs. Fujii’ (about 10 cm in length and about 2.5 cm in width). The branch color of a one-year old plant of ‘Kurachouchou’ is dark green (RHS color chart 152A), whereas the branch color of a one-year old plant of ‘Mrs. Fujii’ is dark red purple, almost brown (RHS color chart 180A). ‘Kurachouchou’ has a smaller truss size (about 18 cm in diameter and about 18 cm in height) than ‘Mrs. Fujii’ (about 25 cm in diameter and about 20 cm in height). ‘Kurachouchou’ has more corolla per truss, about 20, than ‘Mrs. Fujii,’ about 15, and the size of the corolla of ‘Kurachouchou’ is smaller (about 7 cm in length, about 7 cm in height, and about 6 cm in thickness) than ‘Mrs. Fujii’ (about 10 cm in length, about 10 cm in height, and about 5 cm in thickness).

### BRIEF DESCRIPTION OF THE DRAWINGS

The accompanying photographs show typical specimens of the new variety, including foliage and flowers, in color as nearly true as it is reasonably possible to make in color illustrations of these characteristics.

FIG. 1 illustrates the upper side of the flowers and the plant;

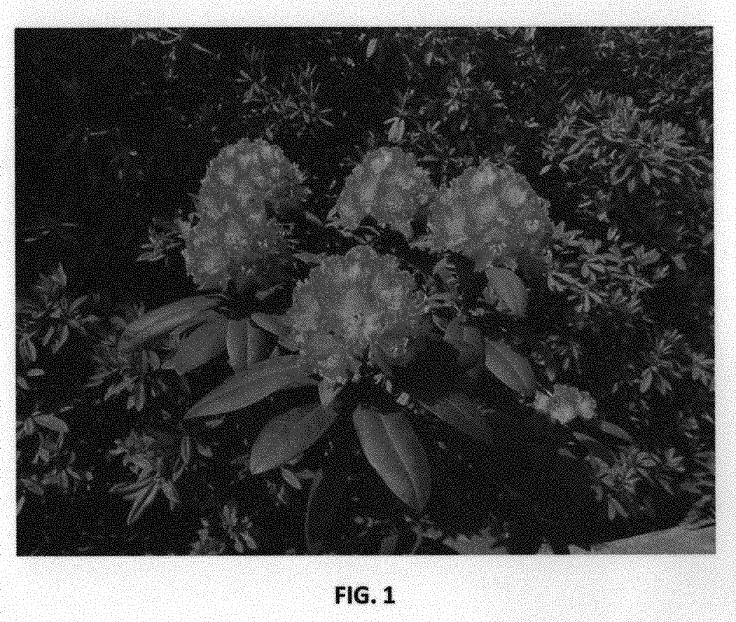

FIG. 2 illustrates a full view of the cultivation area showing the leaves of the plant;

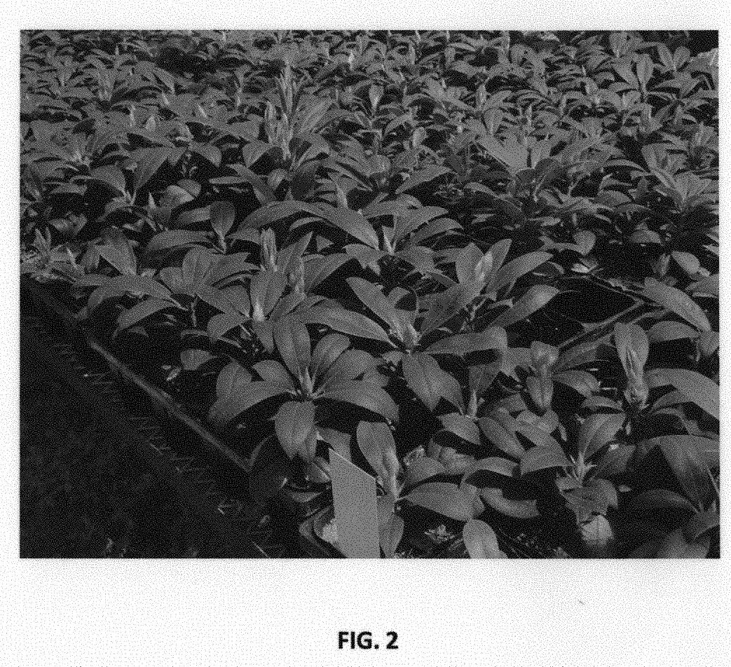

FIG. 3 illustrates a close-up of the flower; and

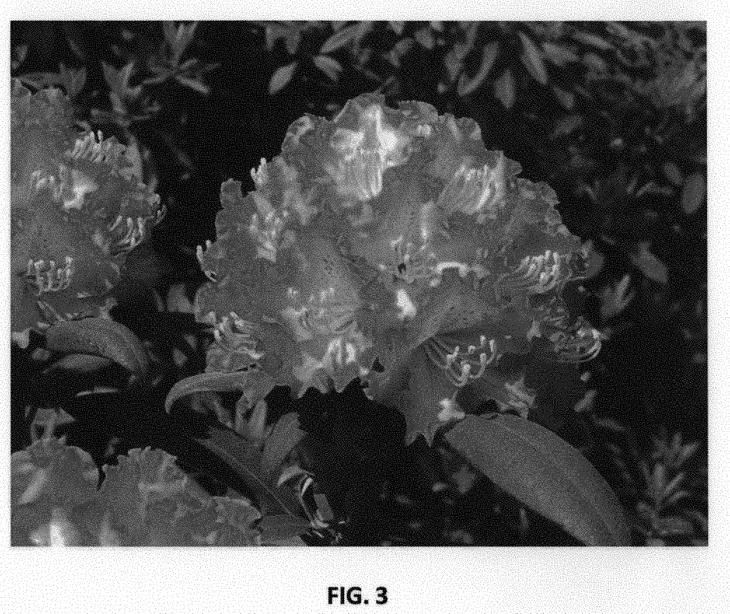

FIG. 4 illustrates, for comparison, the upper side of ‘Mrs. Fujii.’

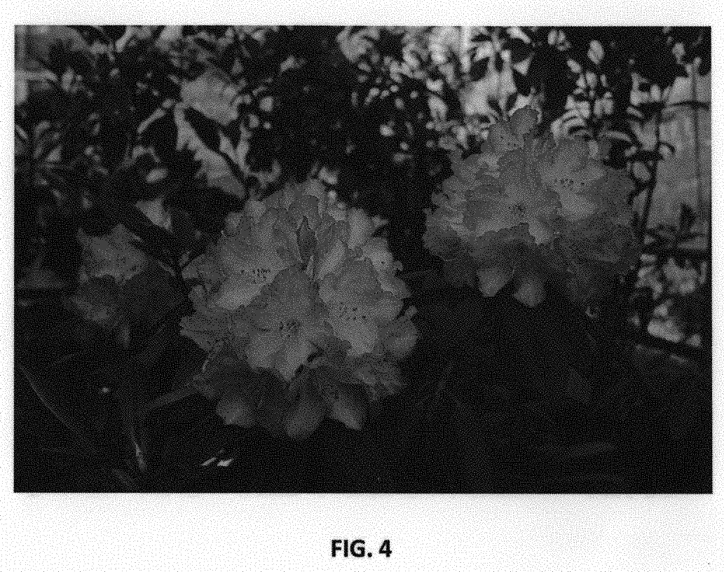

### DESCRIPTION OF THE VARIETY

The following detailed description of the new variety is based upon observations taken of plants grown in Anoucho, Tsu-shi, Mie-ken, Japan. Observations of ‘Kurachouchou’ were taken during the 2015 growing season.

In the following description, color references are made to The Royal Horticultural Society (R.H.S.) Colour Chart, except where general terms of ordinary dictionary significance are used.
Botanical classification: Rhododendron L.
Principle use: Flowering shrub for potted plant and open-field cultivation.
Parentage: The female parent is ‘Stardust,’ and the male parent ‘Pink Ice.’
Propagation: The new variety was propagated using tissue cultures. Growth points were collected from lateral buds of shoots, and cultivation and growth were performed without using calli.
Plant:
   Size.—At 3 years of age, plants of the new variety commonly exhibit a height of about 40-50 cm and a width of about 30 cm.
   Habit.—Vigorous and compact.
Foliage:
   Type.—Evergreen flowering shrub, simple semi-glossy lanceolate leaves with acute apex, and obtuse margins.
   Size.—Mature leaves growing midway up the stem commonly are approximately 18 cm in length and approximately 18 cm in width.
   Color.—RHS color chart NN137B on the upper surface, and RHS color chart 143C on the under surface.
Branches:
   Color.—One-year old wood RHS color chart 152A.
Flowers:
   Period.—Blooms approximately May 5 in the evening when cultured at Anoucho, Tsu-shi, Mie-ken, Japan.
   Truss shape.—Generally a sphere.
   Truss size.—Approximately 18 cm diameter, and approximately 18 cm in height.
   Corolla per truss.—Approximately 20.
   Corolla shape.—Open funnel shape.
   Corolla size.—Approximately 7 cm in length, 7 cm in width, and 6 cm in thickness.
   Number of lobes.—Five lobes per floret.
   Margin of lobes.—Wavy to frilly.
   Fragrance.—None.
   Calyx size.—Approximately 2 millimeters (mm) in length.
   Color.—Two colors on the surface of the corolla lobe. The principle color on the surface of the corolla lobe is strong purplish red (RHS color chart 71C). The secondary color on the surface of the corolla lobe is very light purple (RHS color chart 76B). Weak, independent spots are present on the throat of the corolla lobe.
Winter hardiness: Has survived winters with temperatures as low as −3° C. in Anoucho, Tsu-shi, Mie-ken, Japan.
Disease resistance: During observations to date, the foliage has been disease resistant. Although generally, Rhododendron L. plants are prone to root rot disease, which tends to occur in in high temperatures, ‘Kurachouchou’ is resistant to root rot because it is resistant to high temperatures.

What is claimed is:

1. A new and distinct variety of Rhododendron L. plant, substantially as shown and described.
